(19) United States
(12) Patent Application Publication
(10) Pub. No.: US 2024/0408372 A1
(43) Pub. Date: Dec. 12, 2024

(54) Cap for Intraoperative Suction Device

(71) Applicant: Baptist Health South Florida, Inc., Coral Gables, FL (US)

(72) Inventors: Domenech Asbun, Coral Gables, FL (US); Horacio Asbun, Coral Gables, FL (US)

(21) Appl. No.: 18/740,779

(22) Filed: Jun. 12, 2024

Related U.S. Application Data
(60) Provisional application No. 63/507,562, filed on Jun. 12, 2023.

(51) Int. Cl. A61M 39/20; A61M 39/00

(52) U.S. Cl. CPC A61M 39/20; A61M 2039/0009

(57) ABSTRACT

A cap configured to be mounted to a medical irrigation or suction tube includes a proximal end configured to be connected to the suction tube including a proximal opening having a first diameter and a distal end including a distal opening having a second diameter that is smaller than the first diameter. The cap also includes a sidewall extending between the proximal end and the distal end. A portion of the sidewall includes at least one protrusion configured to engage a fenestration in a sidewall of the suction tube for securing the cap to the irrigation or suction tube.

### CROSS REFERENCE TO RELATED APPLICATION

The present application claims the benefit of U.S. Provisional Patent Application No. 63/507,562, filed Jun. 12, 2023, the disclosure of which is hereby incorporated by reference in its entirety.

### BACKGROUND OF THE INVENTION

### Field of the Invention

The present disclosure is generally directed to devices, such as covers, caps, chisels, blades, and similar tools, configured to be mounted to suction tubes and other catheters, as well as to suction tube assemblies and methods for preforming suction procedures, such as laparoscopy.

### Description of Related Art

Laparoscopic surgical devices, which can include laparoscopes, trocars, closure devices, suction/irrigation devices, insufflation devices, robot-assisted surgery systems, and hand access instruments, are surgical tools designed to enable doctors to perform precise laparoscopic surgeries for various medical conditions. The use of these instruments allows for laparoscopic surgeries to be minimally invasive, usually leaving less than half-inch incisions on the area being operated on. Also, these devices allow for less bleeding and pain after the surgeries, improving surgical outcomes for patients. The instruments are available in a variety of sizes, shapes, materials, and designs to provide surgeons with the ability to perform various surgical techniques.

Minimally invasive laparoscopic surgical techniques, for example abdominal laparoscopic surgery, have gained popularity in recent decades. Suction tubes are frequently used in laparoscopic surgery to suck out blood and debris from a surgical site and to irrigate the surgical site in order to keep the site visually clear. A typical suction tube has fenestrations or holes at the working end of the tube through which both suction and irrigation are applied. Different manufacturers use similar designs, which can comprise a long, cylindrical plastic or metal suction tube, with the fenestrations at its tip. The suction tube can be controlled by a handle on an opposite proximal end of the tube. The surgeon holds the handle and controls either aspiration or irrigation by, for example, pressing an activation button on the handle. These aspiration techniques can be particularly useful during complex operations with inflammation, scarring, and obliteration of anatomic planes.

During such minimally invasive procedures, surgeons often use the laparoscopic instrument (e.g., a suction/irrigation device, such as a suction tube) for blunt dissection of tissues, which can be referred to as suction dissection. During such procedures, the negative pressure provided by the suction both improves visualization by removing fluid during dissection and also engages the tissue into the suction tip, which applies additional force against the tissue where dissection is desired. However, in most cases, the suction tubes used during such procedures are not designed for use in soft tissue dissection. Therefore, using such suction tubes may require the surgeon to apply more pressure to the tube to dissect tissue, than would be required if using a more precise instrument. The increased pressure can lead to blunt tears in the soft tissue.

### SUMMARY OF THE INVENTION

According to an aspect of the present disclosure, a cap configured to be mounted to a medical suction or irrigation tube includes: a proximal end configured to be connected to the tube including a proximal opening having a first diameter; a distal end including a distal opening having a second diameter that is smaller than the first diameter; and at least one sidewall extending between the proximal end and the distal end. A portion of the sidewall includes at least one protrusion configured to engage a fenestration in a sidewall of the suction tube for securing the cap to the suction tube.

According to another aspect of the present disclosure, a suction or irrigation tube assembly includes a suction or irrigation tube including a proximal end, an open distal end, an annular sidewall extending therebetween, and at least one fenestration extending through the sidewall. The assembly also includes any of the previously described caps, with the at least one protrusion of the cap extending through the at least one fenestration of the suction tube, thereby removably securing the cap to the suction tube.

According to another aspect of the present disclosure, a kit of parts includes a suction or irrigation tube with a proximal end configured to be connected to a suction and/or irrigation source, a distal end opposite the proximal end, a sidewall extending between the proximal end and the distal end, and at least one fenestration extending through the sidewall. The kit also includes a plurality of the any of the previously described caps configured to be removably mounted to the distal end of the suction tube. The first diameter of the plurality of caps varies so that the plurality of caps can be connected to different sizes of suction tubes.

According to another aspect of the present disclosure, a method of using a surgical tool during a surgical procedure includes attaching a cap, such as any of the previously described caps, to a distal end of a suction tube. The method further includes: connecting a proximal end of the suction tube to a suction source; contacting tissue with the cap for cutting, dissecting, or impinging the tissue; and activating the suction source to draw tissue and fluids into a lumen of the suction tube through the distal opening of the cap.

According to another aspect of the present disclosure, a method of using a surgical tool during a surgical procedure includes attaching one of the previously described caps to a distal end of an irrigation tube; connecting a proximal end of the irrigation tube to a fluid source; activating the fluid source causing the fluid to flow through the irrigation tube towards the cap; and positioning the cap to direct a stream of the fluid expelled from the cap towards tissue at a surgical site.

Non-limiting illustrative examples of embodiments of the present disclosure will now be described in the following numbered clauses.

Clause 1: A cap configured to be mounted to a medical suction or irrigation tube, comprising: a proximal end configured to be connected to the suction or irrigation tube comprising a proximal opening having a first diameter; a distal end comprising a distal opening having a second diameter that is smaller than the first diameter; and at least one sidewall extending between the proximal end and the distal end, wherein a portion of the sidewall comprises at least one protrusion configured to engage a fenestration in a sidewall of the suction tube for securing the cap to the suction tube.

Clause 2: The cap of clause 1, comprising a metal, such as stainless steel, tempered steel, hardened steel, high carbon steel, titanium, or aluminum.

Clause 3: The cap of clause 1 or clause 2, wherein a portion of the sidewall of the cap defines a conical or partially conical annular surface configured for contacting soft tissue.

Clause 4: The cap of any of clauses 1-3, wherein the distal end of the cap comprises a sharpened tip at least partially enclosing the distal opening.

Clause 5: The cap of any of clauses 1-4, wherein the distal end of the cap comprises a chisel or blade for dissecting soft tissue.

Clause 6: The cap of any of clauses 1-5, wherein an outer diameter of the proximal end of the cap matches or substantially matches an inner diameter of the suction tube.

Clause 7: The cap of clause 6, wherein the at least one protrusion extends outward from an outer surface of the sidewall of the cap.

Clause 8: The cap of clause 7, wherein the sidewall is outwardly biased, thereby pressing the at least one protrusion through the fenestration of the suction tube.

Clause 9: The cap of any of clauses 1-8, wherein the first diameter of the proximal opening matches or substantially an outer diameter of the suction tube.

Clause 10: The cap of clause 9, wherein the at least one protrusion extends inward from an inner surface of the sidewall.

Clause 11: The cap of clause 10, wherein the sidewall is inwardly biased, thereby pressing the at least one protrusion through one of the fenestrations of the suction tube.

Clause 12: The cap of any of clauses 1-11, wherein the at least one protrusion comprises a plurality of axially aligned protrusions.

Clause 13: The cap of any of clauses 1-12, wherein the at least one protrusion comprises at least one first protrusion on one side of the cap and at least one second protrusion on another side of the cap, separated from the first protrusion by 180 degrees.

Clause 14: The cap of any of clauses 1-13, wherein the at least one protrusion comprises a plurality of protrusions arranged circumferentially about the sidewall of the cap.

Clause 15: The cap of any of clauses 1-14, wherein the at least one protrusion comprises a post having a first end connected to the sidewall and an opposing second end, and wherein a shape of a cross-section of the post matches a shape of the fenestration of the suction tube.

Clause 16: The cap of any of clauses 1-15, wherein the proximal opening and the distal opening are concentric, with the proximal opening and the distal opening each centered on a longitudinal axis of the cap and/or the suction tube.

Clause 17: The cap of any of clauses 1-16, wherein the distal opening is a circle or ellipse.

Clause 18: The cap of any of clauses 1-17, wherein the distal opening comprises a slit.

Clause 19: The cap of any of clauses 1-18, wherein the distal end of the cap comprises an annular conical surface with the distal opening enclosed by the annular conical surface.

Clause 20: The cap of any of clauses 1-19, wherein the distal end of the cap comprises a plurality of distal openings extending through the sidewall of the cap.

Clause 21: The cap of any of clauses 1-20, wherein the sidewall comprises a first longitudinal edge and a second longitudinal edge, and wherein the cap is formed by folding the first longitudinal edge over the second longitudinal edge.

Clause 22: The cap of clause 21, wherein the first longitudinal edge and the second longitudinal edge are not fixed together, so that the first diameter of the proximal opening can be adjusted by moving the first longitudinal edge relative to the second longitudinal edge.

Clause 23: A suction or irrigation tube assembly, comprising: a suction or irrigation tube comprising a proximal end, an open distal end, an annular sidewall extending therebetween, and at least one fenestration extending through the sidewall; and the cap of any of clauses 1-22, wherein the at least one protrusion of the cap extends through the at least one fenestration of the suction tube, thereby removably securing the cap to the suction tube.

Clause 24: The assembly of clause 23, wherein the proximal end of the suction or irrigation tube comprises a port configured to be connected to a suction source or a source of fluid for irrigation of a surgical site.

Clause 25: A kit of parts comprising: a suction or irrigation tube comprising a proximal end configured to be connected to a suction and/or irrigation source, a distal end opposite the proximal end, a sidewall extending between the proximal end and the distal end, and at least one fenestration extending through the sidewall; and a plurality of the caps of any of clauses 1-23 configured to be removably mounted to the distal end of the suction tube, wherein the first diameter of the plurality of caps varies, so that the plurality of caps can be connected to different sizes of suction tubes.

Clause 26: The kit of clause 25, wherein, for some of the plurality of caps, the at least one protrusion extends inwardly from an inner surface of the sidewall and, for others of the plurality of caps, the at least one protrusion extends outward from an outer surface of the sidewall.

Clause 27: The kit of clause 25 or clause 26, wherein the plurality of caps comprises at least one cap with the distal opening comprising a slot, another at least one cap with a plurality of the distal openings, and another at least one cap comprising a circular distal opening that is concentric with the proximal opening.

Clause 28: The kit of any of clauses 25-27, wherein at least one of the plurality of caps comprises a conical annular surface enclosing the distal opening with the distal opening being a circle enclosed by the conical annular surface, and another of the plurality of caps comprises opposing longitudinal edges that are folded together to form the cap.

Clause 29: A method of using a surgical tool during a surgical procedure, comprising attaching the cap of any of clauses 1-23 to a distal end of a suction tube; connecting a proximal end of the suction tube to a suction source; contacting tissue with the cap for cutting, dissecting, or impinging the tissue; and activating the suction source to draw the tissue and fluids into a lumen of the suction tube through the distal opening of the cap.

Clause 30: The method of clause 29, wherein the surgical procedure comprises a minimally invasive laparoscopic procedure.

Clause 31: The method of clause 29 or clause 30, further comprising selecting the cap to be used for the surgical procedure from a kit comprising a plurality of the caps of any of clauses 1 to 23.

Clause 32: The method of any of clauses 29-31, wherein applying suction comprises applying varying amounts of suction.

Clause 33: A method of using a surgical tool during a surgical procedure, comprising attaching the cap of any of clauses 1-23 to a distal end of an irrigation tube; connecting a proximal end of the irrigation tube to a fluid source; activating the fluid source causing the fluid to flow through the irrigation tube towards the cap; and positioning the cap to direct a stream of the fluid expelled from the cap towards tissue at a surgical site.

Clause 34: The method of clause 33, further comprising selecting the cap to be used for the surgical procedure from a kit comprising a plurality of the caps of any of clauses 1 to 23.

### BRIEF DESCRIPTION OF THE DRAWINGS

These and other features and characteristics of the present disclosure, as well as the methods of operation and functions of the related elements of structures and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limit of the invention.

FIG. 1A is a front view of a cap for a suction tube, according to an example of the present disclosure;

FIG. 1B is a top view of the cap of FIG. 1A;

FIG. 1C is a cross-sectional view of the cap of FIG. 1A;

FIG. 2A is a front view of another example of a cap for a suction tube, according to an example of the present disclosure;

FIG. 2B is a top view of the cap of FIG. 2A;

FIG. 2C is a cross-sectional view of the cap of FIG. 2A;

FIG. 3A is a front view of another example of a cap for a suction tube, according to an example of the present disclosure;

FIG. 3B is a top view of the cap of FIG. 3A;

FIG. 3C is a schematic drawing shown the cap of FIG. 3A prior to assembly, according to an example of the disclosure;

FIGS. 4A and 4B are top views of additional examples of caps for suction tubes, according to examples of the present disclosure;

FIG. 5A is a schematic drawing showing an assembly including a suction tube and a cap, according to an example of the present disclosure;

FIG. 5B is a schematic drawing of a cross-sectional view of the assembly of FIG. 5A;

FIG. 5C is a schematic drawing of a cross-sectional view of an assembly including a cap inserted into a distal end of a suction tube, according to an example of the present disclosure; and

FIG. 6 is a schematic drawing of a kit including a suction tube and multiple caps, according to an example of the present disclosure.

### DESCRIPTION OF THE INVENTION

As used herein, the singular form of “a”, “an”, and “the” include plural referents unless the context clearly states otherwise.

As used herein, the terms “right”, “left”, “top”, “bottom”, and derivatives thereof shall relate to the invention as it is oriented in the drawing figures. As used herein, the term “proximal” refers to a portion of a tool or device that is grasped by a user and/or is farthest away from a patient. For example, a “proximal” end of a catheter can refer to an end of the catheter that is held by a practitioner or surgeon and/or connected to a fluid delivery device. By contrast, the term “distal” refers to an end of a tool or device that is opposite the proximal end. The distal end of the device can be an end that is farthest away from the portion grasped by the user. For example, for an indwelling catheter, the distal end can be the end which is inserted into the patient through an insertion site. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

For the purposes of this specification, unless otherwise indicated, all numbers expressing, for example, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention. Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “comprising,” “comprise” or “comprised,” and variations thereof, are meant to be open ended. As used herein, the term “patient” or “subject” refers to members of the animal kingdom including but not limited to human beings.

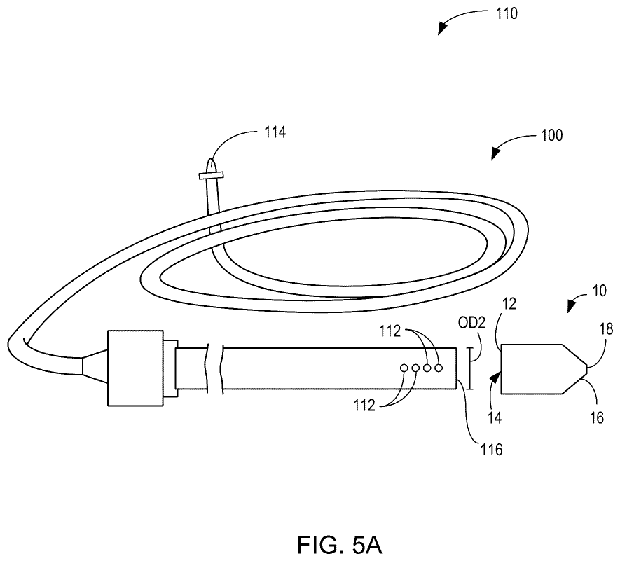

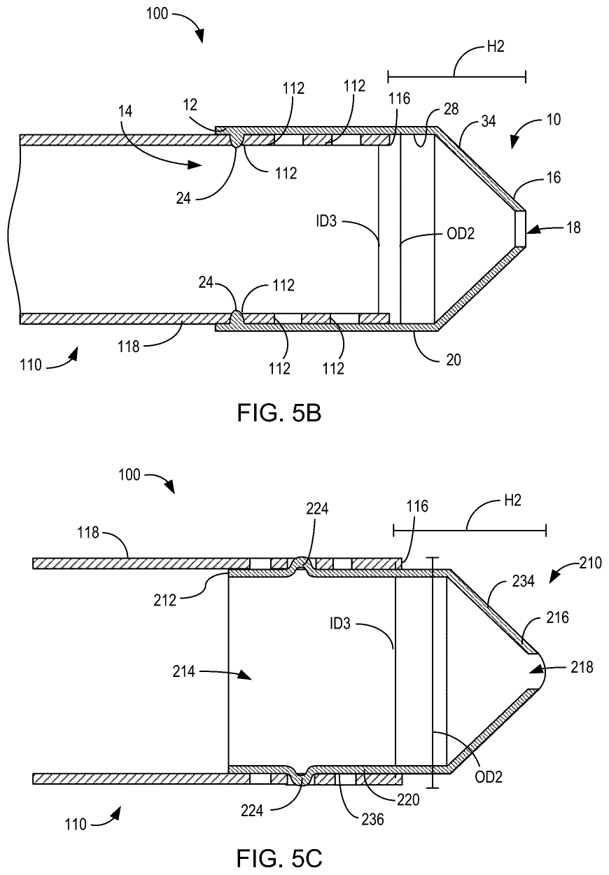

With reference to the figures, the present disclosure is directed to surgical tools and devices, such as caps 10, 210, 310 configured to be connected to a suction/irrigation tool (e.g., an irrigation or suction tube 110, shown in FIGS. 5A-5C). The present disclosure is also directed to assemblies 100 including caps 10, 210, 310 connected to other medical devices, such as catheters or suction tubes 110, as well as to kits 102 with multiple caps 10, 210, 310 configured to be attached to different types of suction tubes 110 and/or configured for use in different tissue dissection techniques. The caps 10, 210, 310 disclosed herein are intended to provide an alternative to current techniques, where surgeons use a distal end of the suction tube 110 itself for tissue dissection or cutting. As previously described, tissue dissection and cutting with the current style suction tube 110 and/or head can make surgeries less safe and timely by requiring the surgeon to apply substantial pressure to the suction tube 110 to achieve a desired result. Moreover, unwanted problems can arise when surgeons use currently available suction tubes 110 in a makeshift way. In particular, as previously described, surgeons often use laparoscopic instruments, such as suction tubes 110, for blunt dissection of tissues (“suction dissection”), even though these instructions are not designed for this use. For example, in current techniques, the open end of a metal suction tube 110 can be pushed against tissue, while simultaneously varying degrees of suction are applied, which aids in the identification of tissue planes and separation of tissues. This approach requires more pressure, which can lead to blunt tears in the tissue.

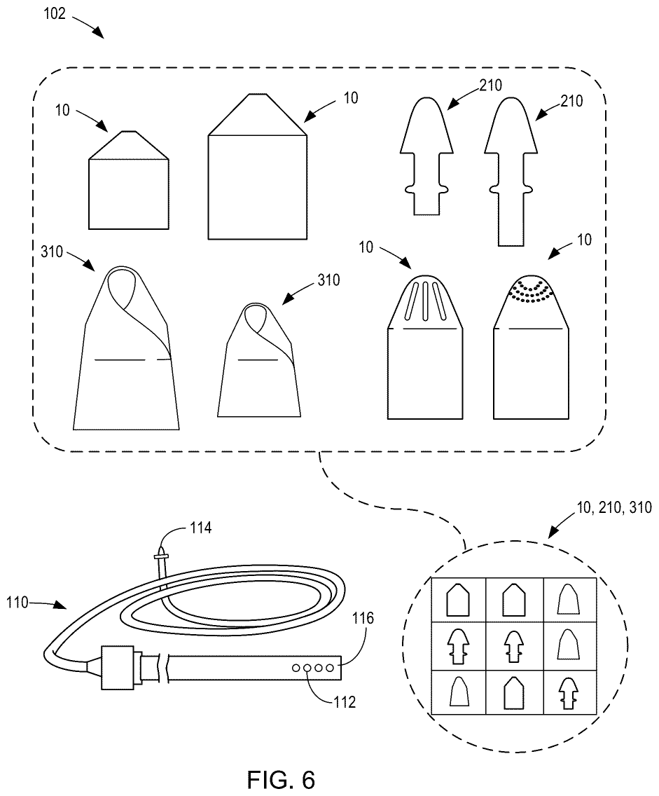

The caps 10, 210, 310 and other surgical tools disclosed herein are configured to attach to conventional irrigation or suction tubes 110, providing a narrowed suction outlet that gives the surgeon more control over a direction of suction force applied through a suction tube 110. The caps 10, 210, 310 can also be configured to provide a narrow fluid stream for irrigation. In particular, the caps 10, 210, 310 can allow a surgeon to precisely direct a stream of pressurized fluid towards particular tissue of a surgical site, which can aid in tissue dissection (e.g., for “hydro-dissection”). As shown in FIGS. 5A and 6, a conventional irrigation or suction tube 110 can include a rigid tube or hollow rod, such as a rigid tube formed from metal having a cylindrical cross section. The rigid tube can be attached to flexible tubing having a proximal end configured to be connected to a suction source and/or a source of fluid for irrigation and a distal end connected to the rigid tube. The irrigation or suction tube 110 can also include an activation button or valve for controlling when suction and/or irrigation is provided through the rigid tube.

In some examples, the cap 10, 210, 310 can comprise a tip placed at the operating end of the suction tube 110. The cap 10, 210, 310 can narrow to a distal opening providing a more focused and controllable suction and dissection force. In some examples, the caps 10, 210, 310 disclosed herein are configured to attach onto suction devices (e.g., suction tubes 110) used during abdominal laparoscopic surgeries. The caps 10, 210, 310 allow for more controlled and meticulous application of pressure directly at a desired point or location. Further, the caps 10, 210, 310 disclosed herein do not reduce the suction device's ability to provide irrigation. While generally intended for use in minimally invasive surgical procedures, in some examples, the caps 110, 210, 310 disclosed herein can also be modified for attachment to handheld suction devices for use during open surgery.

In some examples, the caps 10, 210, 310 are configured to be removably mounted over the relatively large circular opening of the suction tube 110, thereby replacing the large opening with a smaller opening. The smaller opening can be shaped like a chisel, pencil, or similar round surface and opening. Desirably, the caps 10, 210, 310 disclosed herein can be used with suction tubes 110 and catheters from a variety of manufacturers, provided that the suction tubes 110 have fenestrations at a working end thereof, for engaging the cap 10, 210, 310.

In some examples, the caps 10, 210, 310 disclosed herein address issues of inadvertent tearing, by including a more precise distal chisel-tip point design. This design allows for less pressure to be used when dissecting smaller sections of tissue. It is believed that applying less pressure to soft tissues reduces a risk that adjacent tissues, which do not need to be dissected, will tear or be damaged during surgical procedures.

In some examples, the distal end of the cap 10, 210, 310 can be modified into other distal shapes. For example, the cap 10, 210, 310 can be made to resemble a pencil tip, curved chisel, or any other convenient shape, granting the surgeon greater options when performing laparoscopic procedures and allowing for use of fewer attachments and tools on other dissecting devices.

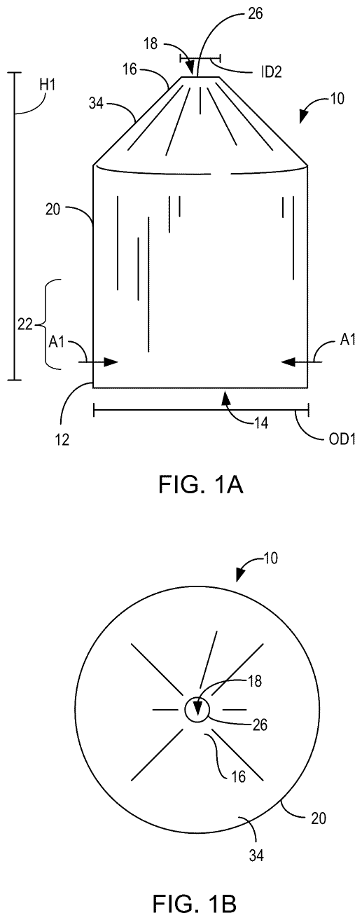

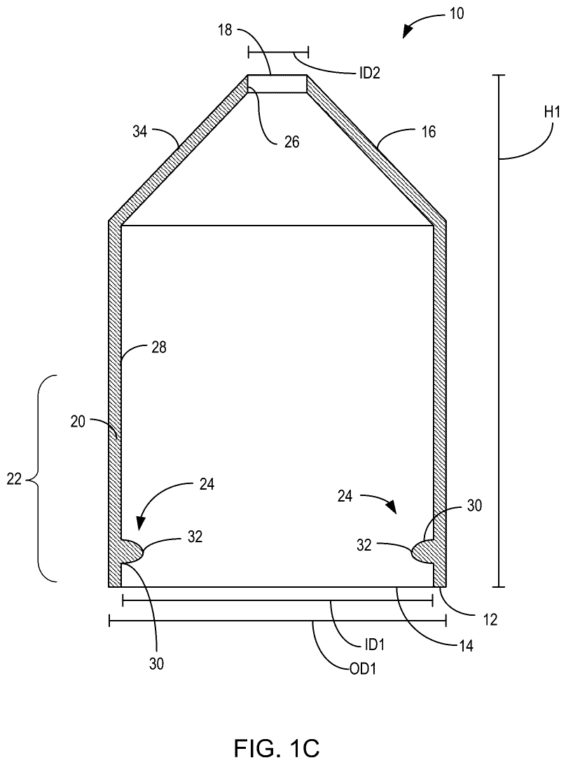

With reference to FIGS. 1A-IC, in some examples, a cap 10 configured to be mounted to a medical suction tube 110 (shown in FIGS. 5A-5C), such as to a substantially rigid tube or hollow rod portion of the suction tube 110, comprises a proximal end 12 defining a proximal opening 14 configured to be connected to the suction tube 110, a distal end 16 defining a distal opening 18, and a sidewall 20 extending between the proximal end 12 and the distal end 16. The sidewall 20 of the cap 10 includes an attachment portion 22 configured to be connected to the suction tube 110, which comprises one or multiple protrusions 24 configured to engage a fenestration 112 through a sidewall of the suction tube 110 for securing the cap 10 to the suction tube 110. The cap 10 can be formed from a rigid material, such as a rigid plastic, or a metal, such as stainless steel, tempered steel, hardened steel, high carbon steel, titanium, or aluminum.

The proximal end 12 of the cap 10 includes or defines the proximal opening 14 having a first or proximal diameter ID1 (shown in FIG. 1C). The proximal diameter ID1 is selected so that the cap 10 can be mounted to a distal end 116 (shown in FIGS. 5A-5C) of the suction tube 110. Therefore, the proximal diameter ID1 is generally selected to match a standard diameter of commercially available suction tubes. For example, the proximal diameter ID1 of the proximal opening 14 can be about 3.0 mm to about 8.0 mm or, preferably, about 5.0 mm. An outer diameter OD1 of the proximal end 12 of the cap 10 can be about 4.0 mm to about 9.0mm or, preferably, about 6.0 mm.

The distal opening 18 at the distal end 16 of the cap 10 is narrower than the proximal opening 14 in order to focus and restrict the suction force provided through the cap 10. For example, the distal opening 18 can have a second or distal diameter ID2 of about 2.0 mm to about 4.0 mm. The cap 10 can have a total height H1 (e.g., a distance between the proximal end 12 and the distal end 16) of about 10.0 mm to about 40.0 mm. Further, when the cap 10 is connected to the suction tube 110, the distal end 16 of the cap 10 can be configured to extend beyond the distal end 116 of the suction tube 110 by a distance H2 (shown in FIGS. 5B and 5C) of about 5.0 mm to about 30.0 mm depending upon the shape of the cap 10.

In some examples, as shown in FIGS. 1A-1C, a portion of the sidewall 20 of the cap 10 defines a conical or partially conical annular surface 34 configured for contacting soft tissue. For example, a portion of the sidewall 20 of the cap 10 can form a conical or tapered annular surface 34 enclosing the distal opening 18. The cap 10 can also include a sharpened tip. For example, the annular edge 26 enclosing the distal opening 18 can be sharpened to enhance tissue dissection provided by the cap 10. The distal end 16 of the cap 10 can also be made form a structure for impinging or cutting soft tissue. For example, the distal end 16 of the cap 10 can comprise a chisel or blade for dissecting the soft tissue.

In some examples, as shown in FIGS. 1A-1C, the cap 10 is configured to be inserted over the distal end of the suction tube 110. In such examples, the proximal diameter ID1 of the proximal opening 14 of the cap 10 can be made to match or substantially match the outer diameter OD2 (shown in FIGS. 5A-5C) of the suction tube 110. As used herein, a diameter of the cap 10 “matches” a diameter of a corresponding portion of the suction tube 110, when the diameter of the cap 10 is equal to the diameter of the corresponding portion of the suction tube 110. The diameter of the cap 10 “substantially matches” the diameter of the suction tube 110, when the diameter of the cap 10 is within about 10% of the diameter of the corresponding portion of the suction tube 110. For caps 10 that are configured to be inserted over the distal end 116 of the suction tube 110, the protrusion 24 extends inward from an inner surface 28 of the sidewall 20 of the cap 10. Further, the sidewall 20 of the cap 10 can be inwardly biased, as shown by arrows A1 in FIG. 1A, in order to press the protrusion 24 through one of the fenestrations 112 of the suction tube 110.

The caps 10 of the present disclosure can have a variety of arrangements of protrusions 24 configured to be pressed into the fenestrations 112 of the suction tube 110, selected to match different fenestration arrangements of commercially available suction tubes 110. In general, the protrusions 24 can be posts having a first end 30 connected to the sidewall 20 of the cap 10 and an opposing second end 32. A shape of a cross-section of the protrusion 24 or post can match or substantially match (e.g., having a cross-sectional area that is within about 10% of) a shape of the fenestration 112 of the suction tube 110. The shape of the cross-section can be, for example, a circle, oval, square, rectangle, or another convenient shape. In some examples, the cap 10 can include a plurality of axially aligned protrusions 24, such as two, three, four, or more protrusions, configured to be inserted into axially aligned fenestrations 112 of the suction tube 110. In other examples, as shown in FIG. 1C, the cap 10 can include protrusions 24 aligned about a circumference of the sidewall 20 of the cap 10. For example, the cap 10 can include a first protrusion 24 extending inwardly from the inner surface 28 on one side of the cap 10 and a second protrusion 24 extending inwardly from the inner surface 28 of the sidewall 20 on an opposite side of the cap 10. The first protrusion 24 can be spaced apart from the second protrusion 24 by about 180 degrees.

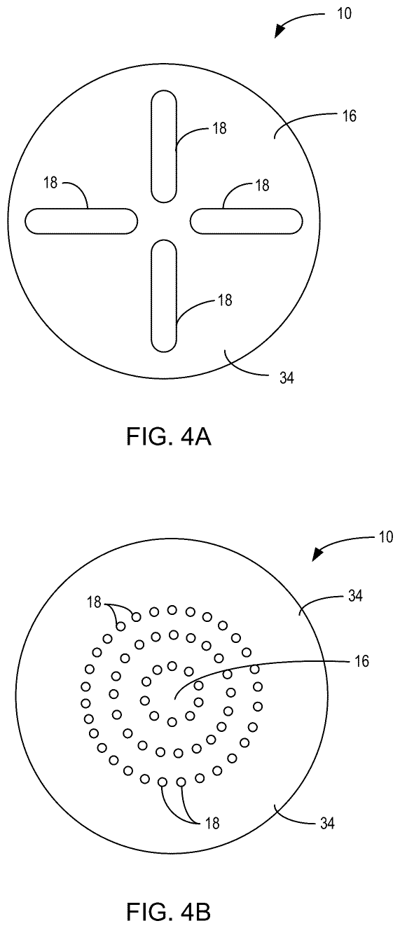

The cap 10 can also include a variety of different examples of distal openings 18 for providing suction and irrigation from the suction tube 110 to soft tissue. In some examples, as shown in FIGS. 1A-1C, the proximal opening 14 and the distal opening 18 are concentric, with the proximal opening 14 and the distal opening 18 each centered on a longitudinal axis of the cap 10 and/or the suction tube 110. Further, the distal opening 18 can be a circle (as shown in FIGS. 1A-1C), an ellipse, or any other convenient shape. As shown in FIG. 4A, the distal opening 18 can include a slit, such as a slit extending through the annular tapered or conical surface 34 of the cap 10. The slit can be an axially aligned slit, extending from the distal end 16 of the cap 10 towards the proximal end 12. The slit can also be helical, curving about a central longitudinal axis of the cap 10. In other examples, as shown in FIG. 4B, the cap 10 can include multiple distal openings 18 on the distal end 16 of the cap 10. For example, the conical or substantially conical annular surface 34 can include multiple perforations extending through the sidewall 20 of the cap 10. The multiple perforations can be arranged in any convenient and easily manufactured pattern, such as in concentric rings aligned with or centered on a longitudinal axis of the cap 10. In other examples, the perforations can be arranged in rows and columns or as a spiral.

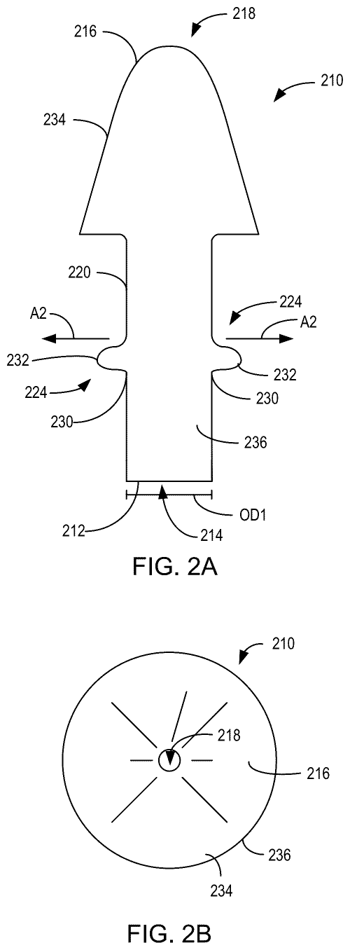

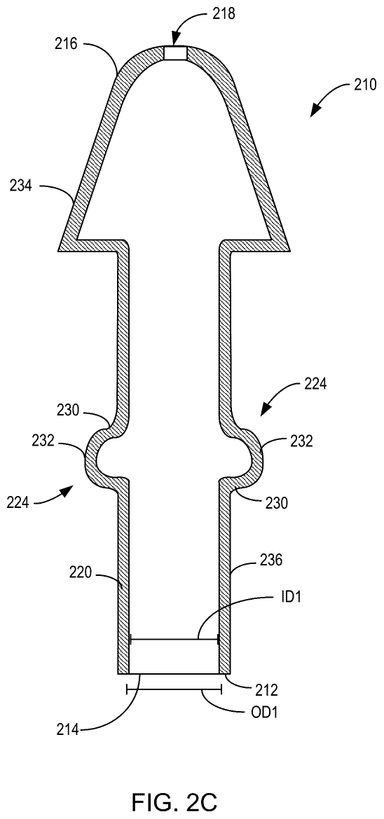

FIGS. 2A-2C show another example of a cap 210 configured to be mounted to a medical suction tube, such as the suction tube 110 shown in FIGS. 5A-5C. As in previous examples, the cap 210 comprises the proximal end 212 configured to be connected to the suction tube 110 having a proximal opening 214 having a first diameter ID1, the distal end 216 comprising a distal opening 218 having a second diameter ID2 that is smaller than the first diameter ID1, and the sidewall 220 extending between the proximal end 212 and the distal end 216. As in previous examples, the cap 210 can comprise and/or can be formed from a rigid material, such as rigid plastic or metal, such as stainless steel, tempered steel, hardened steel, high carbon steel, titanium, or aluminum.

Unlike in previous examples, the cap 210 shown in FIGS. 2A-2C is configured to be inserted into the open distal end 116 of the suction tube 110 (shown in FIGS. 5A-5C). In such configurations, the cap 210 can be configured such that an outer diameter OD1 of the proximal end 212 of the cap 210 matches or substantially matches (e.g., is within 10% of) an inner diameter ID3 (shown in FIGS. 5A-5C) of the suction tube 110. For example, the outer diameter OD1 of the cap 210 can be about 3.0 mm to about 5.0 mm or, preferably, about 4.0 mm.

Further, unlike in previous examples, the protrusions 224 of the cap 210 extend outward from an outer surface 236 of the sidewall 220 of the cap 210. For example, the protrusions 224 can comprise a cylindrical post having a first end 230 connected to or integral with the outer surface 236 of the sidewall 220 and a second end 232 opposite the first end 230. The posts or protrusions 224 can extend radially outward from other portions of the cap 210 by a distance of about 0.25 mm to about 1.0 mm meaning that an outer diameter of a portion of the cap 210 including the protrusions 224 can be about 3.5 mm to about 7.0 mm. The protrusions 224 are configured to be inserted into fenestrations 112 of the suction tube 110 for securing the cap 210 to the suction tube 110. In such instances, the sidewall 220 of the cap 210 can be biased outwardly, as shown by arrows A2 in FIG. 2A, in order to press the protrusions 224 through the fenestrations 112 of the suction tube 110.

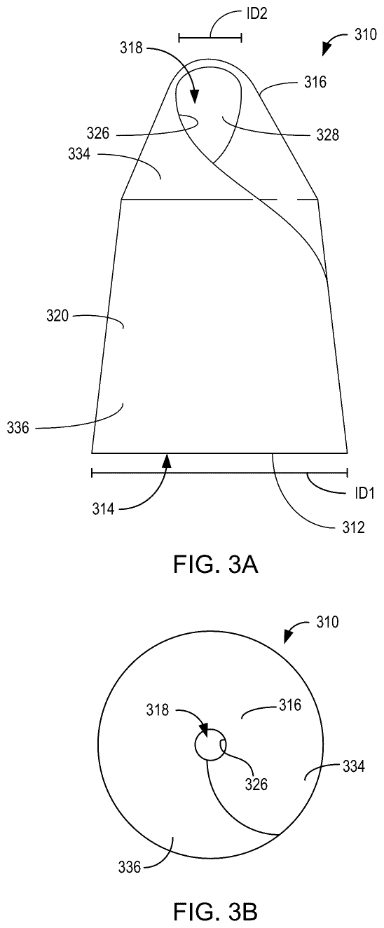

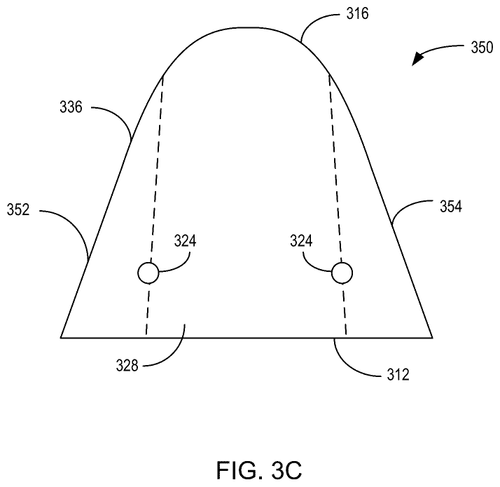

FIGS. 3A-3C show another example of a cap 310 configured to be mounted to a medical suction tube, such as the suction tube 110 shown in FIGS. 5A-5C. As in previous examples, the cap 310 comprises the proximal end 312 configured to be connected to the suction tube 110 having a proximal opening 314 with a first diameter ID1, the distal end 316 having a distal opening 318 with a second diameter ID2 that is smaller than the first diameter ID1, and the sidewall 320 extending between the proximal end 312 and the distal end 316. As in previous examples, the inner proximal diameter ID1 can be about 3.0 mm to about 8.0 mm or, preferably, about 5.0 mm. The inner distal diameter ID2 can be about 2.0 mm to about 4.0 mm. Also as in previous examples, the cap 310 can comprise and/or can be formed from a rigid material, such as rigid plastic or metal, such as stainless steel, tempered steel, hardened steel, high carbon steel, titanium, or aluminum.

Unlike in previous examples, the cap 310 is formed from a sheet 350 (shown in FIG. 3C) that is rolled, folded, or bent to form the cap 310. For example, the sheet 350 can include a first longitudinal edge 352 on one side of the sheet 350 and a second longitudinal edge 354 on an opposing side of the sheet 350. The cap 310 can be formed by folding the first longitudinal edge 352 over the second longitudinal edge 354 in order to form an annular structure sized to connect to the distal end 116 of the suction tube 110. In some examples, the first longitudinal edge 352 and the second longitudinal edge 354 are not fixed together. In such configurations, the diameter ID1 of the proximal opening 314 of the cap 310 can be adjusted by sliding the first longitudinal edge 352 relative to the second longitudinal edge 354 to increase or decrease the overlap between longitudinal edges 352, 354 of the sheet 350, thereby increasing or decreasing the proximal diameter ID1 of the proximal opening 314 to accommodate suction tubes 110 of different shapes and sizes. In other examples, the first longitudinal edge 352 can be fixed to the second longitudinal edge 354 by, for example, an adhesive or mechanical fastener. In such cases, the proximal opening 314 of the cap 310 is a fixed size selected to be connected to a specific size of suction tube 110.

As in previous examples, the cap 310 can also include the one or more protrusions 324 configured to engage corresponding fenestrations 112 of the suction tube 110 for securing the cap 310 to the suction tube 110. The protrusions 324 can extend from an inner surface 328 of the sidewall 320 for a cap 310 configured to be inserted over the distal end 116 of the suction tube 110 and/or from an outer surface 336 of the sidewall 320 for a cap 310 configured to be inserted into the distal end 116 of the suction tube 110.

As in previous examples, the distal end 316 of the cap 310 can include a sharpened or pointed tip for dissecting, cutting, or impinging soft tissue. For example, the cap 310 can include a sharpened edge 326 around the distal opening 318. In some examples, the edge 326 can be bent or curved to form a chisel for puncturing or cutting soft tissue. Alternatively or in addition, the sidewall 320 of the cap 310 can be bent or manipulated to form a conical or partially conical annular surface 334 enclosing the distal opening 318. The conical annular surface 334 can be configured to form a pointed tip for contacting soft tissue.

FIGS. 5A-5C show a suction tube assembly 100 comprising an irrigation or suction tube 110 or catheter and a cap 10, 210, 310 configured to be removably connected to the suction tube 110. As previously described, the irrigation or suction tube 110 can include a distal portion including a rigid tube or hollow rod and a proximal portion formed from flexible tubing configured to be connected to a suction or irrigation source. The suction tube 110 can be a commercially available suction tube, such as any of a variety of tubes manufactured by Fairmont Medical Products Pty Ltd, Stryker Corporation, Medtronic Plc., and others.

The suction tube 110 can include a proximal end 114, such as a proximal end of the flexible tubing, which can include a port configured to be connected to a suction source for applying suction to soft tissue and/or a fluid source for irrigation, and a distal end 116, such as a distal end of the rigid tube or rod, configured to removably receive the cap 10, 210, 310. In some examples, the rigid tube or rod can include a cylindrical sidewall 118 extending from a proximal end of the rod to the distal end 116. The sidewall 118 includes or defines one or more fenestrations 112 or holes extending through the sidewall 118 in fluid communication with a lumen of the rigid tube or rod. Generally, the fenestrations 112 are positioned near the distal end 116 of the rigid tube or rod, such as within from about 4.0 mm to about 20.0 mm of the distal end 116 of the suction tube 110. Other portions of the rigid tube or rod and suction tube 110 can be free from fenestrations 112 and/or from other holes, perforations, or openings through the sidewall 118 of the suction tube 110. The fenestrations 112 can be any size determined, for example, based on the suction force intended to be provided by the suction source through the lumen of the suction tube 110. In some examples, the fenestrations 112 can have a diameter or maximum dimensions of about 0.5 mm to about 2.0 mm. Further, each of the fenestrations 112 can have an area of about 0.2 mm² to about 4.0 mm².

In some examples, as previously described, the rigid tube or rod can include multiple fenestrations 112. For example, the rigid tube or rod can include two, four, six, eight, or more fenestrations 112. The total area of the multiple fenestrations can be about 0.5 mm² to about 50.0 mm². The multiple fenestrations 112 can be axially aligned, extending from the distal end 116 towards the proximal end of the rigid tube or rod. Alternatively or in addition, the rigid tube or rod can include fenestrations 112 aligned about a circumference of the suction tube 110, such as a first fenestration 112 on one side of the rigid tube or rod and a second fenestration 112 on another side of the rigid tube or rod separated from the first fenestration 112 by, for example, about 180 degrees.

The rigid tube or rod can be formed from metal, such as stainless steel, tempered steel, hardened steel, high carbon steel, titanium, or aluminum. The suction tube 110 can also include the flexible tubing extending from the proximal end of the rigid tube or rod. The flexible tubing can be formed from any flexible or bendable polymer material, such as silicone, polyester, polyethylene, or acrylonitrile butadiene styrene.

FIG. 6 shows a kit 102 of parts for use in a suction procedure, such as a laparoscopic surgical procedure. The kit 102 includes the irrigation or suction tube 110. As previously described, the suction tube 110 can be a commercially available suction tube or catheter from any of a variety of manufacturers. The suction tube 110 includes the flexible tubing comprising the proximal end 114 configured to be connected to a suction source and/or to a fluid source for irrigation. The suction tube 100 also includes the rigid tube or rod comprising the distal end 116 configured to be attached to the cap 10 and the sidewall 118 extending between a proximal end of the rigid tube or rod and the distal end 116. As in previous examples, the rigid tube or rod of the suction tube 110 includes one or multiple fenestrations 112 extending through the sidewall 118 of the rigid tube or rod for providing the suction force and/or irrigation fluid to target locations.

The kit 102 also includes multiple caps 10, 210, 310 of different dimensions and/or including different features, which can be removably connected to the distal end 116 of the rigid tube or rod. For example, the multiple caps 10, 210, 310 can have proximal openings 14, 214, 314 with different proximal diameters IDI so that the caps 10, 210, 310 can be connected to different sizes of suction tubes 110.

The multiple caps 10, 210, 310 can also include caps 10, 210, 310 with different arrangements of protrusions 24, 224, 324 configured to be connected to rigid tubes or rods with different arrangements of fenestrations 112. For example, some of the caps 10, 310 can be configured to be inserted over the open distal end 116 of the rigid tube or rod. Such caps 10, 310 include protrusions 24, 324 extending inwardly from an inner surface 28, 328 of the sidewall 20, 320 of the cap 10, 310. Other caps 210 can be configured to be inserted into the distal end 116 of the rigid tube or rod. Such caps 210 have protrusions extending outwardly from an outer surface 236 of the sidewall 220 of the cap 210. Further, some of the caps 310 can be formed from a sheet 350 including the first longitudinal edge 352 folded over the second longitudinal edge 354. Such caps 310 can be adjustable allowing the user, such as the surgeon, to adjust the proximal diameter ID1 of the proximal opening 314 by increasing or decreasing an overlap between the first longitudinal edge 352 and the second longitudinal edge 354.

The multiple caps 10, 210, 310 can also include caps 10, 210, 310 with different arrangements and configurations of distal openings 18, 218, 318. For example, some of the caps 10, 210, 310 can include circular distal openings 18, 218, 318. In some examples, the proximal opening 14, 214, 214 and the distal opening 18, 218, 318 of the caps 10, 210, 310 can be concentric, with the proximal opening 14, 214, 314 aligned with the distal opening 18, 218, 318 along a longitudinal axis of the cap 10, 210, 310 and/or the suction tube 110. Other caps 10 (shown in FIG. 4A) can include a distal opening 18 comprising an elongated slot or slit. Other caps 10 (shown in FIG. 4B) can include multiple distal openings 18 or perforations, such as distal openings 18 arranged in rows and columns over a conical or tapered annular surface 34 of the sidewall 20 of the cap 10. The practitioner or surgeon can select which of the multiple caps 10, 210, 310 to use for a particular procedure and, when ready to perform the procedure, can attach the selected cap 10, 210, 310 to the distal end 116 of the suction tube 110.

More specifically, when using the suction tube assemblies 100 and caps 10, 210, 310 of the present disclosure for performing a surgical method, the surgeon or another trained medical professional first selects the cap 10, 210, 310 to be used for a particular procedure from multiple caps 10, 210, 310 provided, for example, as a kit 102 (shown in FIG. 6) of surgical tools. For example, the surgeon's selection may be based on a size of the cap 10, 210, 310, size of the suction tube 110 to which the cap 10, 210, 310 will be connected, and/or a type of surgical procedure to be performed. In particular, a cap 10, 210, 310 with a sharpened tip or distal edge may be selected when a surgical procedure to be performed requires cutting and/or dissecting tissue. Caps 10, 210, 310 with more blunt and/or less sharp surfaces may be selected when the suction tube 110 is only being used for directed suction and/or irrigation, and not for dissecting tissue.

Once the cap 10, 210, 310 is selected, in performing the surgical method, the surgeon next attaches the cap 10, 210, 310 to the distal end 116 of the rigid tube or rod of the suction tube 110. For example, the cap 10, 310 can be inserted over the distal end 116 of the rigid tube or rod causing protrusions 24, 324 extending inwardly from an inner surface 28, 328 of the sidewall 20, 320 of the cap 10, 310 to insert through the fenestrations 112 of the rigid tube or rod. Alternatively, the cap 210 can be inserted into the distal end 116 of the rigid tube or rod, causing protrusions 224 extending outwardly from an outer surface 236 of the sidewall 220 to insert through the fenestrations 212 of the rigid tube or rod.

The method can also include a step of attaching a proximal end 114 of the suction tube 110 to a suction source, such as a portable or free-standing vacuum pump or to a wall suction outlet of a hospital or medical facility. Alternatively or in addition, the proximal end 114 of the suction tube 110 can be connected to a fluid source, such as a source of saline solution, for providing irrigation fluid through the lumen of the suction tube 110.

When ready to perform the surgical procedure, such as a minimally invasive laparoscopic procedure, the surgeon can insert the distal end 116 of the rigid tube or rod of the suction tube 110 and cap 10, 210, 310 attached thereto into the patient through an incision, such as a small incision in the abdomen or pelvic region of the patient. The surgeon can contact soft tissue within the abdomen and/or pelvic region with the cap 10, 210, 310 for dissecting, cutting, or impinging the soft tissue. While contacting the soft tissue, the suction source can be activated for suction of fluids proximate to the soft tissue and/or for irrigation. Suction force applied by the suction source can also draw soft tissue towards the distal opening(s) 16, 216, 316 of the cap 10, 210, 310 enhancing the tissue dissecting and cutting ability of the cap 10, 210, 310. In some examples, a varying suction force can be applied through the lumen of the suction tube 110, which aids in the identification of tissue planes and separation of tissues. In some examples, fluid can also be applied to a target site through the suction tube 110 for irrigation of the target site.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. Furthermore, it is to be understood that the present invention contemplates that, to the extent possible, one or more features of any embodiment can be combined with one or more features of any other embodiment

The invention claimed is:

1. A cap configured to be mounted to a medical suction or irrigation tube, comprising:
a proximal end configured to be connected to the suction or irrigation tube comprising a proximal opening having a first diameter;
a distal end comprising a distal opening having a second diameter that is smaller than the first diameter; and
at least one sidewall extending between the proximal end and the distal end,
wherein a portion of the at least one sidewall comprises at least one protrusion configured to engage a fenestration in a sidewall of the suction or irrigation tube for securing the cap to the suction or irrigation tube.

2. The cap of claim 1, comprising at least one of stainless steel, tempered steel, hardened steel, high carbon steel, titanium, or aluminum.

3. The cap of claim 1, wherein a portion of the sidewall of the cap defines a conical or partially conical annular surface configured for contacting soft tissue.

4. The cap of claim 1, wherein the distal end of the cap comprises a sharpened tip at least partially enclosing the distal opening.

5. The cap of claim 1, wherein the distal end of the cap comprises a chisel or blade for dissecting soft tissue.

6. The cap of claim 1, wherein the at least one protrusion extends outward from an outer surface of the at least one sidewall of the cap, and
wherein the at least one sidewall is outwardly biased, thereby pressing the at least one protrusion through the fenestration of the suction or irrigation tube.

7. The cap of claim 1, wherein the first diameter of the proximal opening matches or substantially an outer diameter of the suction or irrigation tube.

8. The cap of claim 7, wherein the at least one protrusion extends inward from an inner surface of the at least one sidewall.

9. The cap of claim 8, wherein the at least one sidewall is inwardly biased, thereby pressing the at least one protrusion through one of the fenestrations of the suction or irrigation tube.

10. The cap of claim 1, wherein the at least one protrusion comprises a post having a first end connected to the at least one sidewall and an opposing second end, and wherein a shape of a cross-section of the post matches a shape of the fenestration of the suction or irrigation tube.

11. The cap of claim 1, wherein the distal opening comprises a slit.

12. The cap of claim 1, wherein the at least one sidewall comprises a first longitudinal edge and a second longitudinal edge, and wherein the cap is formed by folding or rolling the first longitudinal edge over the second longitudinal edge.

13. A suction or irrigation tube assembly, comprising:
a suction or irrigation tube comprising a proximal end, an open distal end, an annular sidewall extending therebetween, and at least one fenestration extending through the annular sidewall; and
the cap of claim 1, wherein the at least one protrusion of the cap extends through the at least one fenestration of the suction or irrigation tube, thereby removably securing the cap to the suction or irrigation tube.

14. A kit of parts comprising:
a suction or irrigation tube comprising a proximal end configured to be connected to a suction and/or irrigation source, a distal end opposite the proximal end, a sidewall extending between the proximal end and the distal end, and at least one fenestration extending through the sidewall; and
a plurality of the caps of claim 1 configured to be removably mounted to the distal end of the suction or irrigation tube, wherein the first diameter of the plurality of caps varies, so that the plurality of caps can be connected to different sizes of suction tubes.

15. The kit of claim 14, wherein, for some of the plurality of caps, the at least one protrusion extends inwardly from an inner surface of the sidewall and, for others of the plurality of caps, the at least one protrusion extends outward from an outer surface of the sidewall.

16. The kit of claim 14, wherein the plurality of caps comprises at least one cap with the distal opening comprising a slot, another at least one cap with a plurality of the distal openings, and another at least one cap comprising a circular distal opening that is concentric with the proximal opening.

17. The kit of claim 14, wherein at least one of the plurality of caps comprises a conical annular surface enclosing the distal opening with the distal opening being a circle enclosed by the conical annular surface, and another of the plurality of caps comprises opposing longitudinal edges that are folded together to form the cap.

18. A method of using a surgical tool during a surgical procedure, comprising
attaching the cap of claim 1 to a distal end of a suction tube;
connecting a proximal end of the suction tube to a suction source;
contacting tissue with the cap for cutting, dissecting, or impinging the tissue; and
activating the suction source to draw the tissue and fluids into a lumen of the suction tube through the distal opening of the cap.

19. The method of claim 18, wherein the surgical procedure comprises a minimally invasive laparoscopic procedure.

20. The method of claim 18, further comprising selecting the cap to be used for the surgical procedure from a kit comprising a plurality of the caps of claim 1.
